(19) United States
(12) Patent Application Publication
(10) Pub. No.: US 2004/0241217 A1
(43) Pub. Date: Dec. 2, 2004

(54) Method for administration of immune modulators in systemic and localized immune disorders

(76) Inventors: Yee-Chien Liu, Road Town, VI (US); Shiou-Ru Tzeng, Road Town, VI (US); Chi-Hui Huang, Road Town, VI (US); Jya-Wei Cheng, Road Town, VI (US)

Correspondence Address: ROSENBERG, KLEIN & LEE, 3458 ELLICOTT CENTER DRIVE-SUITE 101, ELLICOTT CITY, MD, 21043, US

(21) Appl. No.: 10/446,676

(22) Filed: May 29, 2003

(51) Int. Cl. A61K 38/13; A61K 31/60; A61K 31/4745; A61K 9/70

(52) U.S. Cl. 424/449; 514/11; 514/165; 514/291

(57) ABSTRACT

The present invention provides an immune modulator administration method whereby the modulators are formulated into a patch or cream to help patients in managing the diseases and alleviate the immune syndromes. The patch comprises a baking layer, a penetration enhancing layer, a carrier layer and a pressure sensitive adhesive layer. The application of immune modulators onto a topical patch or cream renders a method in the management of immune disorders either systemic or localized. The invention also provides clinicians a convenient mechanism for monitoring therapy responses. Furthermore, utilization of the present invention improves the quality of life for the living being to which the immune modulators are administrated. The concept and formulations of the present invention dramatically improve effectiveness of the treatment of autoimmune diseases such as psoriasis, rheumatoid arthritis, systematic lupus erythematosus, autoimmune hepatitis, hepatitis B and hepatitis C.

### BACKGROUND OF THE INVENTION

1. Field of the Invention

The present invention relates to a medicinal administration method. More specifically, the present invention discloses a topical or transdermal immune modulator administration method whereby the modulators are formulated into a patch or cream to help the patients in managing the diseases and alleviate the immune syndromes.

2. Description of the Prior Art

An imbalance in the immune system often leads to diseases such as autoimmune syndromes and virus infected hepatitis. Although modern medications cannot absolutely control immune response, some immune modulators are used to alleviate the suffering of patients. These modulators include aspirin, methotrexate hydrate, cyclosporine A, hydrochloroquine, azathioprine, tacrolimus and ascomycin. All of these have been clinically applied in humans for many years in order to treat autoimmune syndromes.

Currently, aspirin is one of the safest and least expensive pain relievers with over 100 years of proven and effective treatment for a variety of ailments. In the late 1800s, salicylate was the standard drug for the treatment of arthritis. However, such treatment was very irritating to the stomach. Subsequently, Hoffman synthesized acetylsalicylic acid (ASA) and then aspirin was created. Aspirin may prevent the cells of the immune system from maturing and attacking the human organs by inhibiting cyclooxygenase 2, which is a key enzyme for producing prostaglandin.

Methotrexate is used to treat rheumatoid arthritis, psoriatic arthritis, Reiter's syndrome, and other conditions. Methotrexate, by altering the body's use of folic acid, was approved for use in adults by the U.S. Food and Drug Administration (FDA) in 1988.

Cyclosporine is the primary tool to prevent transplantation rejection. In addition, the ability of cyclosporine to inhibit T-cell activation plays a role in the treatment of nephrotic syndrome, refractory Crohn's disease and ulcerative colitis, biliary cirrhosis, aplastic anemia, rheumatoid arthritis, myasthenia gravis, and dermatomyositis. Hydrochloroquine, an antimalarial drug effective against joint pain, is a disease-modifying anti-rheumatic drug (DMARD) and is commonly used in the treatment of arthritis.

Azathioprine, an immunosuppressant, disrupts the synthesis of DNA and RNA. This means that it targets cells or tissues with relatively rapid cell division. In the treatment of immune mediated diseases, the stimulated lymphocytes become the prime target of azathioprine.

Tacrolimus, belonging to immunosuppressive macrolides, is obtained from cultures of Streptomyces. Tacrolimus, like cyclosporine and other immunosuppressants, inhibits the secretion of certain proteins which are necessary for the T-cell to function.

Ascomycin macrolactams belong to a new group of immunosuppressive, immunomodulatory and anti-inflammatory agents. Due to their chemical nature they are water-soluble and therefore, in principle, suitable for topical treatment of dermatological disorders However, most drugs are administrated orally and sometimes the active ingredients are destroyed by GI pH variations.

As a result, there is need of a more effective method for utilizing immune modulators for managing systemic and local immune disorders.

Therefore, the present invention provides a topical or transdermal immune modulator administration method whereby the modulators are formulated into a patch or cream to help the patients in managing the diseases are formulated into a patch or cream to help the patients in managing the diseases and alleviate the immune syndromes.

Utilization of the present invention avoids destruction of the active ingredients by GI pH variations and allows further avoidance of first-pass effects by lumen intestinal enzymes. The immune modulators which are formulated into a patch or a cream provide a new way and concept in managing the immune syndromes with a lower dosage and organ toxicity

### SUMMARY OF THE INVENTION

To achieve these and other advantages and in order to overcome the disadvantages of the conventional method in accordance with the purpose of the invention as embodied and broadly described herein, the present invention provides an immune modulator administration method whereby the modulators are formulated into a patch or cream to help the patients in managing the diseases and alleviate the immune syndromes.

The method for administration of immune modulators in systemic and localized immune disorders of the present invention involves the application of various topical formulations. The immune modulators being the active ingredients in the topical compositions may be of any volume and are not limited to a specific range due to the different conditions of the compositions, penetration enhancers and the like.

The present invention provides a method and compositions for abrogating systemic and localized immune disorders by a patch or a cream.

The patch comprises a baking layer, a penetration enhancing layer, a carrier layer and a pressure sensitive adhesive layer. The polyacrylic pressure sensitive adhesive layer comprises methyl vinyl ether and glyceryl behenate. The penetration enhancer layer is a mixture of water, azone, Dimethyl Sulfoxide (DMSO), ethanol, acetone, propyleneglycol, polyethylene glycol, surfactants and oil. The pharmaceutical carrier layer, containing active ingredients such as aspirin, methotrexate hydrate, cyclosporin A, hydrochloroquine, azothioprine, ascomycin and Tacrolimus, comprises water, nitroglycerine, glycerol and DMSO.

The polyacrylic pressure sensitive adhesive contains methyl vinyl ether and glyceryl behenate. Dissolve all the active ingredients in the carrier and lay the mixture onto the pressure sensitive adhesive layer and let air-dry. Subsequently, lay the different penetration enhancers onto the air-dried layer and cover it with a water-impermeable baking layer to form a prototype patch. Finally, cut the patch in squares of 2×3 cm² as a treated patch.

Accordingly, an object of the present invention is to provide a concept for managing diseases related to immune imbalances by utilizing a patch or a cream during treatment.

Another object is to provide an alternative method for alleviating the inflammatory syndromes through the inhibition of cyclooxygenase activity or the depression of immune activity by aspirin and other immunosuppressants.

A further object is to provide a method for formulating the immune modulators into a patch or a cream to manage the immune diseases.

Utilization of the method of the present invention improves the quality of life for the living being to which the immune modulators are administrated.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

In the drawings,

FIG. 1 is a diagram illustrating a patch of the method for administration of immune modulators in systemic and localized immune disorders according to an embodiment of the present invention; The polyacrylic pressure sensitive adhesive layer (a) is composed of methyl vinyl ether and glyceryl behenate. The penetration enhancer layer (b) is a mixture of water, azone, DMSO, ethanol, acetone, propyleneglycol, polyethylene glycol, surfactants and oil. The pharmaceutical carrier layer (c), containing active ingredients such as aspirin, methotrexate hydrate, cyclosporin A, hydrochloroquine, azothioprine, ascomycin and Tacrolimus, consists of water, nitroglycerine, glycerol and DMSO.

FIG. 2 is a graph illustrating the effect of penetration enhancers of a patch on facilitating the penetration of aspirin according to an embodiment of the present invention;

FIG. 3 is a graph illustrating the effect of penetration enhancers of a patch on facilitating the penetration of methotrexate hydrate according to an embodiment of the present invention;

FIG. 4 is a graph illustrating the effect of penetration enhancers of a patch on facilitating the penetration of cyclosporine A according to an embodiment of the present invention;

FIG. 5 is a graph illustrating the effect of penetration enhancers of a patch on facilitating the penetration of hydrochloroquine according to an embodiment of the present invention;

FIG. 6 is a graph illustrating the effect of penetration enhancers of a patch on facilitating the penetration of azothioprine according to an embodiment of the present invention;

FIG. 7 is a graph illustrating the effect of penetration enhancers of a patch on facilitating the penetration of ascomycin according to an embodiment of the present invention;

FIG. 8 is a graph illustrating the effect of penetration enhancers of a patch on facilitating the penetration of tacrolimus according to an embodiment of the present invention;

FIG. 9 is a table illustrating the enhanced effects of the patch as shown in FIGS. 2-8 according to an embodiment of the present invention; and

FIG. 10 is a table illustrating test results such as measured survival rates and average liver weights according to an embodiment of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

In general, diseases and sicknesses treated by a patch or cream can be categorized into seven groups: skin syndrome, motion sickness, anti-angina, hypertension, smoking cessation, hormone replacement therapy and pain management. The drugs delivered by a patch or cream have several advantages such as the reduction of first-pass effects and GI incompatibility, the sustain of therapeutic drug levels, the permission of self-administration, the improvement of patient compliance, the reduction of side effects, the allowance of drug removal and are virtually non-invasive.

The present invention provides a method and compositions for abrogating systemic and localized immune disorders by a patch or a cream. Clinical observations are provided that demonstrate these immune modulators are effective in managing human immune system. Aspirin has been found to be effective in preventing cardiovascular and inflammatory diseases with a daily dose of 80 to 500 mg. Methotrexate can treat psoriasis with doses of 5 to 15 mg/week orally or 10 to 25 mg/week intravenously. Cyclosporine, a well known immunosuppressant, is often used on transplanted patients with doses of 5 to 15 mg/kg/day orally or 5 to 6 mg/kg/day intravenously. Hydrochloroquine, an anti-malarial drug, can be used to orally treat lupus erythematosus and rheumatoid arthritis with doses of 200 to 400 mg/day and 200 to 600 mg/day, respectively. Azathioprine alleviates immune system with doses of 1 to 5 mg/kg/day orally and intravenously. Ascomycin is a newly invented macrolide with hydrophilic property and is often applied as 1% cream to treat atopic dermatitis. Tacrolimus has nearly 100 times more immune suppression activity than cyclosporine and its clinical doses for transplanted patients are 0.1 to 0.2 mg/kg/day orally or 0.03 to 0.05 mg/kg/day intravenously.

In fact, some damages such as neurotoxicity, nephrotoxicity and hepatoxicity may occur in humans if high doses and long-term treatment of these immune modulators are given. Thus, the low dose and slow release of these drugs by means of a patch or a cream may easily provide a new treatment methodology which effectively emphasizes and brings out the positive attributes of the above immune modulators while minimizing their side effects.

The method for administration of immune modulators in systemic and localized immune disorders according to an embodiment of the present invention involves the application of various topical formulations. The immune modulators being the active ingredients in the topical compositions may be of any volume and are not limited to a specific range due to the different conditions of the compositions, penetration enhancers and the like.

Examples of substances which may be used as the immune modulators in the illustrated embodiments comprise the following: aspirin, methotrexate hydrate, cyclosporine A, hydrochloroquine, azathioprine, ascomycin, tacrolimus and any mixture thereof.

Examples of substances which may be used as the polyacrylic pressure sensitive adhesive in the illustrated embodiments comprise the following: methyl vinyl ether, glyceryl behenate and any mixture thereof.

Examples of substances which may be used as the penetration enhancer in the illustrated embodiments comprise the following: water, azone, DMSO, ethanol, acetone, propyleneglycol, polyethylene glycol, surfactants, oil, petroleum and any mixture thereof.

Examples of substances which may be used as the pharmaceutical carrier in the illustrated embodiments comprise the following: water, nitroglycerine, glycerol, DMSO and any mixture thereof.

Examples of substances which may be used as the co-solvent in the illustrated embodiments comprise the following: starch, vegetable oil and any mixture thereof.

However, these examples of substances which may be used are for example and illustrative purposes. It will be apparent to those skilled in the art that various modifications and variations can be made to the present invention without departing from the scope or spirit of the invention. It is intended that the present invention cover modifications and variations of this invention provided they fall within the scope of the invention and its equivalent.

In order to further illustrate the method of the present invention the following examples are provided for clarity but are not intended to limit the scope of the invention.

Example 1 illustrates suggested topical formulations of immune modulators. A composition (100 ml) of topical immune modulators is a water-in-oil cream and consists of the following ingredients: water 5 ml, azone 5 ml, ethanol 5 ml, glycerol 10 ml, DMSO 1 ml, surfactant (Triton X-100) 1 ml, petroleum 40 g, starch 20 g, vegetable oil 20 ml, nitroglycerine 5 ml, aspirin 10 mg, methotrexate hydrate 5 mg, cyclosporine A 5 mg, hydrochloroquine 5 mg, azathioprine 5 mg, ascomycin 2 mg and tacrolimus 2 mg.

The above ingredients are mixed as follows. First, mix all the immune modulators into glycerol with gentle stirring for 10 min and then add DMSO, nitroglycerine and water into the mixture. Second, add Triton X-100 into the mixture and keep stirring for 5 min. Third, add azone, ethanol and the melted petroleum into the mixture and keep stirring. Forth, heat the mixture to 37° C. and add starch and vegetable oil into the mixture. Fifth, continue mixing until the mixture is homogeneous. Finally, let the mixture cool down and transfer it into a labelled tube.

Example 2 illustrates suggested transdermal formulations of immune modulators.

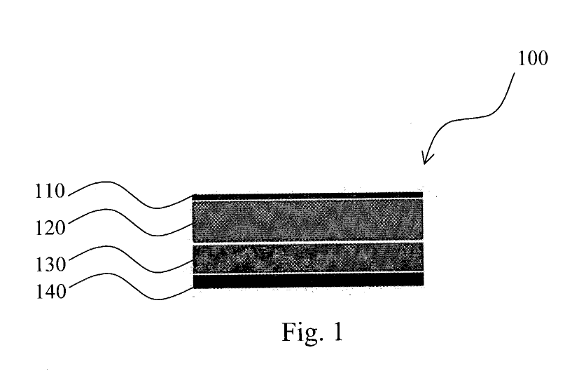

Refer to FIG. 1, which is a diagram illustrating a patch of the method for administration of immune modulators in systemic and localized immune disorders according to an embodiment of the present invention.

The patch comprises a baking layer, a penetration enhancing layer, a carrier layer and a pressure sensitive adhesive layer. The polyacrylic pressure sensitive adhesive layer comprises methyl vinyl ether and glyceryl behenate. The penetration enhancer layer is a mixture of water, azone, Dimethyl Sulfoxide (DMSO), ethanol, acetone, propyleneglycol, polyethylene glycol, surfactants and oil. The pharmaceutical carrier layer, containing active ingredients such as aspirin, methotrexate hydrate, cyclosporine A, hydrochloroquine, azathioprine, ascomycin and Tacrolimus, comprises water, nitroglycerine, glycerol and DMSO.

The detailed formulations of the patch are discussed as follows. The polyacrylic pressure sensitive adhesive contains methyl vinyl ether and glyceryl behenate. Dissolve all the active ingredients (aspirin 100 mg, methotrexate hydrate 50 mg, cyclosporine A 50 mg, hydrochloroquine 50 mg, azathioprine 50 mg, ascomycin 20 mg, tacrolimus 20 mg) in the carrier (70% of water, 10% of nitroglycerine, 10% of glycerol, 10% of DMSO) and lay the mixture onto the pressure sensitive adhesive layer and let air-dry for 10 min. Subsequently, lay the different penetration enhancers (group 1 to group 6) onto the air-dried layer and cover it with a water-impermeable baking layer to form a prototype patch. Finally, cut the patch 100 in squares of 2×3 cm² as a treated patch.

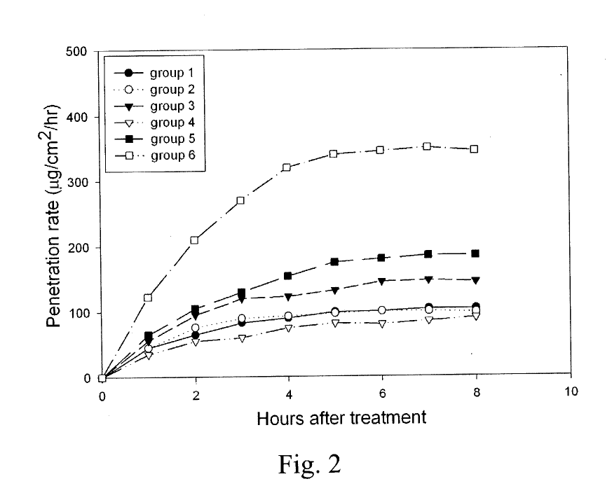

Refer to FIGS. 2-8, which are graphs illustrating the effect of penetration enhancers of a patch on facilitating the penetration of aspirin, methotrexate hydrate, cyclosporine A, hydrochloroquine, azathioprine, ascomycin, and tacrolimus according to embodiments of the present invention.

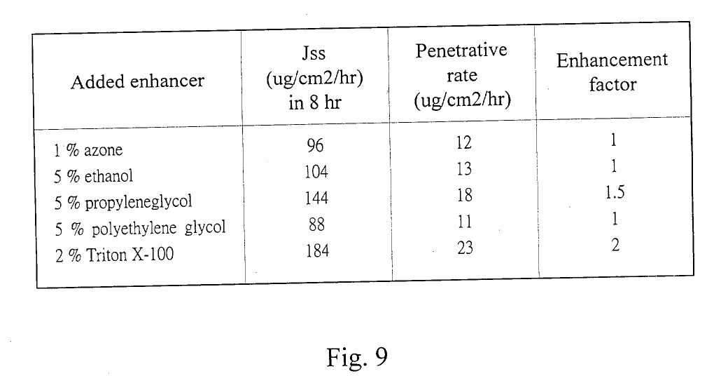

Also, refer to FIG. 9, which is a table illustrating the enhanced effects of the patch as shown in FIGS. 2-8 according to an embodiment of the present invention.

The present invention shows the developments of enhancers by demonstrating the dermal penetrative rate Jss of active ingredients (.mu.g/cm.sup.2/hr) over eight hours. Note: group 1 (1% of azone); group 2 (5% of ethanol); group 3 (5% of propyleneglycol); group 4 (5% of polyethylene glycol); group 5 (2% of Triton X-100); group 6 (1% of azone, 5% of ethanol, 5% of propyleneglycol, 5% of polyethylene glycol, 2% of Triton X-100).

For further detail, refer to FIG. 2, which is a graph illustrating the effect of penetration enhancers of a patch on facilitating the penetration of aspirin according to an embodiment of the present invention.

As shown if FIG. 2, groups 1-6 are plotted in a graph with the penetration rate on the y-axis and the hours after treatment on the x-axis. As clearly demonstrated by the graph, the penetration enhancer mixture of group 6, provides a dramatically improved penetration rate. For example, after 8 hours the penetration rate was approximately 340 as compared with 184 for group 5 and 88 for group 4.

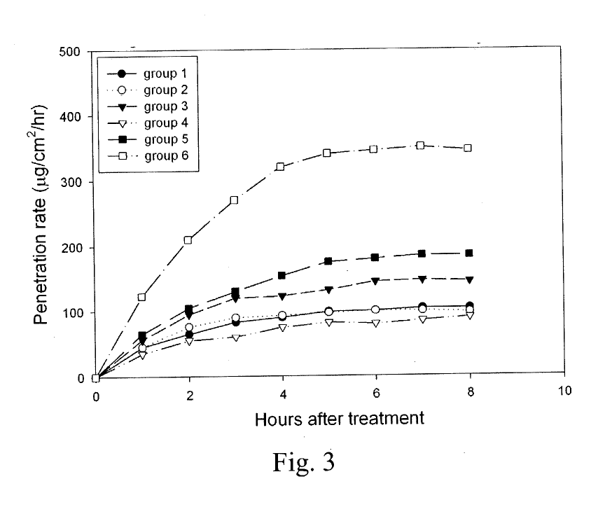

Refer to FIG. 3, which is a graph illustrating the effect of penetration enhancers of a patch on facilitating the penetration of methotrexate hydrate according to an embodiment of the present invention.

As shown if FIG. 3, groups 1-6 are plotted in a graph with the penetration rate on the y-axis and the hours after treatment on the x-axis. As clearly demonstrated by the graph, the penetration enhancer mixture of group 6, provides a dramatically improved penetration rate. For example, after 8 hours the penetration rate was approximately 340 as compared with 184 for group 5 and 88 for group 4.

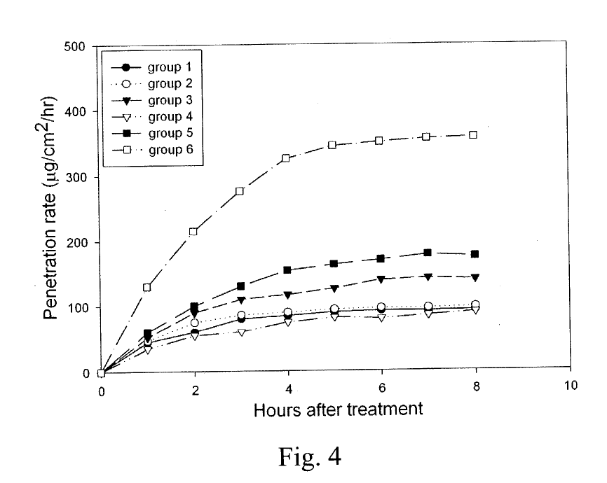

Refer to FIG. 4, which is a graph illustrating the effect of penetration enhancers of a patch on facilitating the penetration of cyclosporine A according to an embodiment of the present invention.

As shown if FIG. 4, groups 1-6 are plotted in a graph with the penetration rate on the y-axis and the hours after treatment on the x-axis. As clearly demonstrated by the graph, the penetration enhancer mixture of group 6, provides a dramatically improved penetration rate. For example, after 8 hours the penetration rate was approximately 350 as compared with 184 for group 5 and 88 for group 4.

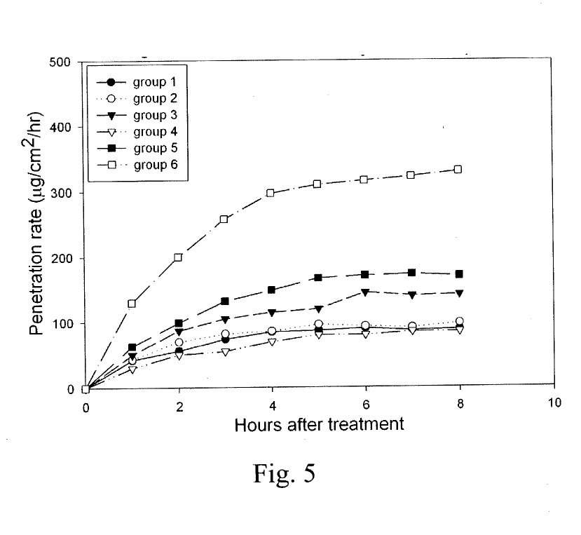

Refer to FIG. 5, which is a graph illustrating the effect of penetration enhancers of a patch on facilitating the penetration of hydrochloroquine according to an embodiment of the present invention.

As shown if FIG. 5, groups 1-6 are plotted in a graph with the penetration rate on the y-axis and the hours after treatment on the x-axis. As clearly demonstrated by the graph, the penetration enhancer mixture of group 6, provides a dramatically improved penetration rate. For example, after 8 hours the penetration rate was approximately 340 as compared with 184 for group 5 and 88 for group 4.

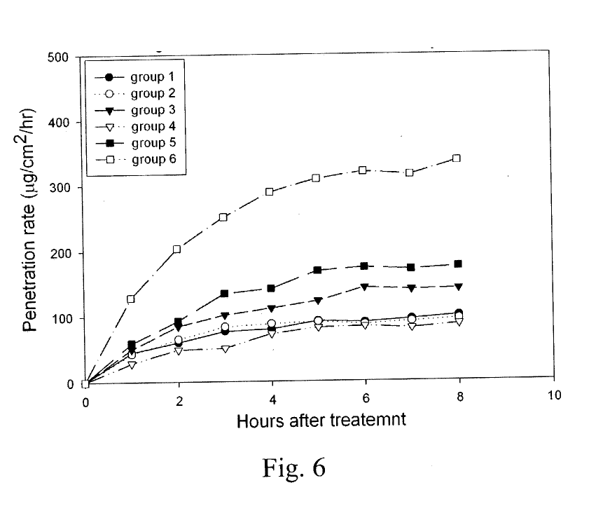

Refer to FIG. 6, which is a graph illustrating the effect of penetration enhancers of a patch on facilitating the penetration of azothioprine according to an embodiment of the present invention.

As shown if FIG. 6, groups 1-6 are plotted in a graph with the penetration rate on the y-axis and the hours after treatment on the x-axis. As clearly demonstrated by the graph, the penetration enhancer mixture of group 6, provides a dramatically improved penetration rate. For example, after 8 hours the penetration rate was approximately 340 as compared with 184 for group 5 and 88 for group 4.

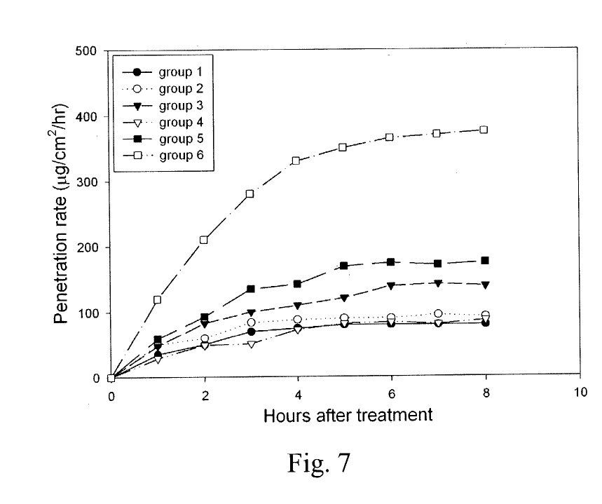

Refer to FIG. 7 is a graph illustrating the effect of penetration enhancers of a patch on facilitating the penetration of ascomycin according to an embodiment of the present invention.

As shown if FIG. 7, groups 1-6 are plotted in a graph with the penetration rate on the y-axis and the hours after treatment on the x-axis. As clearly demonstrated by the graph, the penetration enhancer mixture of group 6, provides a dramatically improved penetration rate. For example, after 8 hours the penetration rate was approximately 360 as compared with 184 for group 5 and 88 for group 4.

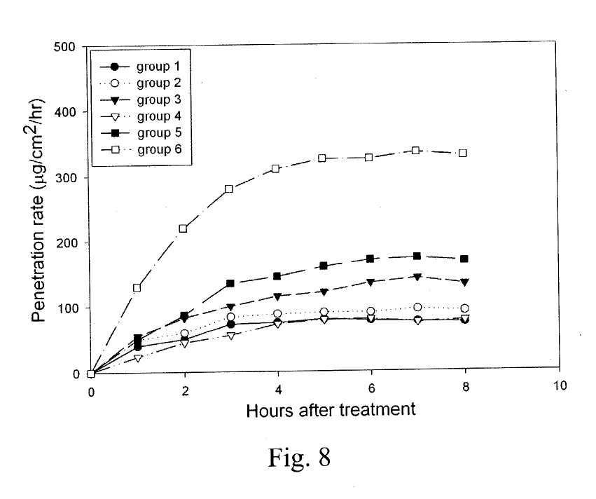

Refer to FIG. 8, which is a graph illustrating the effect of penetration enhancers of a patch on facilitating the penetration of tacrolimus according to an embodiment of the present invention.

As shown if FIG. 8, groups 1-6 are plotted in a graph with the penetration rate on the y-axis and the hours after treatment on the x-axis. As clearly demonstrated by the graph, the penetration enhancer mixture of group 6, provides a dramatically improved penetration rate. For example, after 8 hours the penetration rate was approximately 340 as compared with 184 for group 5 and 88 for group 4.

As clearly illustrated, the immune modulator administration method of the present invention whereby the modulators are formulated into a patch or cream dramatically improves the penetration rate of the modulators in order to help the patients in managing the diseases and alleviate the immune syndromes.

Example 3 illustrates toxicity of transdermal delivery of aspirin, methotrexate hydrate, cyclosporine A, hydrochloroquine, azathioprine, ascomycin and tacrolimus on adult SD mice.

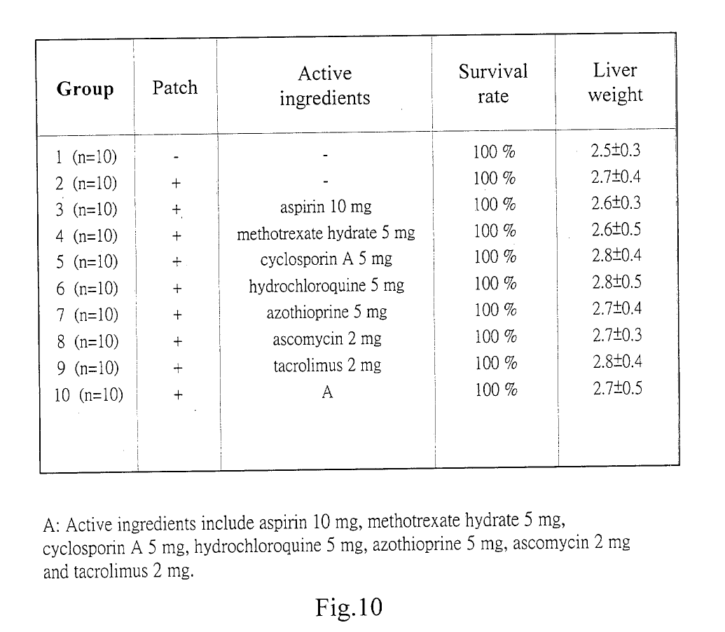

Refer to FIG. 10, which is a table illustrating test results such as measured survival rates and average liver weights according to an embodiment of the present invention.

Note, in FIG. 10, A represents the Active ingredients including aspirin 10 mg, methotrexate hydrate 5 mg, cyclosporine A 5 mg, hydrochloroquine 5 mg, azathioprine 5 mg, ascomycin 2 mg and tacrolimus 2 mg.

Adult males of BALB/cJ mice weighing 120-130 g were obtained. The animals were given standard rat laboratory diet and water ad libitium in a temperature and light controlled room. These animals were divided into different groups and the hair on the back of the animals was removed with a shaver to form a 2.5×3.5 cm² square on each mouse for-treating with immune modulators by a 2×3 cm² patch for 7 days. On the seventh day, the killing effect of immune modulators and the degree of liver damage in the animals were scored by measuring the survival rate and monitoring the changes of liver weight, respectively.

The measured survival rate and average liver weight were 100% and 2.5±0.3 g in group 1 where the animals were not treated with any immune modulator. The survival and average liver weight of the sham operation group where the animals were treated with a patch but without any active ingredient were 100% and 2.7±0.4 g, respectively. As illustrated in FIG. 10, the survival rate in the treated groups was all 100% and the average liver weights were not significantly different from that of group 1. These results indicate that the formulation described in the invention is safe.

As described above, the present invention provides an immune modulator administration method whereby the modulators are formulated into a patch or cream to help the patients in managing the diseases and alleviate the immune syndromes.

Accordingly, the present invention provides a concept for managing diseases related to immune imbalances by utilizing a patch or a cream during treatment.

Additionally, the present invention provides an alternative method for alleviating the inflammatory syndromes through the inhibition of cyclooxygenase activity or the depression of immune activity by aspirin and other immunosuppressants.

Furthermore, the present invention provides a method for formulating the immune modulators into a patch or a cream to manage the immune diseases.

Utilization of the present invention improves the quality of life for the living being to which the immune modulators are administrated.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. A method for treating systemic and localized immune disorders comprising topical or transdermal application of a formulation.

2. The method for treating systemic and localized immune disorders of claim 1, wherein the systemic immune disorders are rheumatoid arthritis, systematic lupus erythematosus, autoimmune hepatitis and virus infected hepatitis.

3. The method for treating systemic and localized immune disorders of claim 1, wherein the localized immune disorders are psoriasis and atopic dermatitis.

4. The method for treating systemic and localized immune disorders of claim 2, wherein the method for treating systemic immune disorder comprises a patch.

5. The method for treating systemic and localized immune disorders of claim 3, wherein the method for treating localized immune disorder comprises a cream.

6. The method for treating systemic and localized immune disorders of claim 4, wherein the patch comprises a polyacrylic pressure sensitive adhesive, a penetration enhancer, and a pharmaceutical carrier.

7. The method for treating systemic and localized immune disorders of claim 6, wherein the pressure sensitive adhesive comprises methyl vinyl ether and glyceryl behenate.

8. The method for treating systemic and localized immune disorders of claim 7, wherein concentration of the pressure sensitive adhesive is between 0.1% and 10%.

9. The method for treating systemic and localized immune disorders of claim 6, wherein the penetration enhancer is a mixture of azone, ethanol, propyleneglycol, polyethylene glycol, and surfactants.

10. The method for treating systemic and localized immune disorders of claim 9, wherein concentration of the penetration enhancer is between 0.05% and 10%.

11. The method for treating systemic and localized immune disorders of claim 6, wherein the pharmaceutical carrier comprises water, nitroglycerine, glycerol and dimethyl sulfoxide.

12. The method for treating systemic and localized immune disorders of claim 11, wherein concentration of the pharmaceutical carrier is between 0.1% and 10%.

13. The method for treating systemic and localized immune disorders of claim 6, wherein immune modulators comprising aspirin, methotrexate hydrate, cyclosporine A, hydrochloroquine, azathioprine, ascomycin and tacrolimus are topically applied.

14. The method for treating systemic and localized immune disorders of claim 13, wherein the immune modulators comprise one or more of the followings: 50-200 mg of aspirin; 10-100 mg of methotrexate hydrate; 20-100 mg of cyclosporine A; 10-100 mg of hydrochloroquine; 10-100 mg of azathioprine; 1-50 mg of ascomycin and 1-50 mg of tacrolimus.

15. The method for treating systemic and localized immune disorders of claim 5, wherein the topically administrated cream comprises a pharmaceutical carrier, a co-solvent and a penetration enhancer.

16. The method for treating systemic and localized immune disorders of claim 15, wherein the pharmaceutical carrier comprises water, nitroglycerine, glycerol and DMSO.

17. The method for treating systemic and localized immune disorders of claim 16, wherein concentration of the pharmaceutical carrier is between 0.1% and 10%.

18. The method for treating systemic and localized immune disorders of claim 15, wherein the co-solvent is a mixture of starch and vegetable oil.

19. The method for treating systemic and localized immune disorders of claim 18, wherein concentration of the co-solvent is between 0.1% and 10%.

20. The method for treating systemic and localized immune disorders of claim 15, wherein the penetration enhancer comprises water, azone, ethanol, surfactants and petroleum.

21. The method for treating systemic and localized immune disorders of claim 20, wherein concentration of the penetration enhancer is between 0.05% and 10%.

22. The method for treating systemic and localized immune disorders of claim 15, wherein immune modulators comprising aspirin, methotrexate hydrate, cyclosporine A, hydrochloroquine, azathioprine, ascomycin and tacrolimus are topically applied.

23. The method for treating systemic and localized immune disorders of claim 22, wherein the immune modulators comprise one or more of the followings: 50-200 mg of aspirin; 10-100 mg of methotrexate hydrate; 20-100 mg of cyclosporine A; 10-100 mg of hydrochloroquine; 10-100 mg of azathioprine; 1-50 mg of ascomycin and 1-50 mg of tacrolimus.
